(19) United States
(12) Patent Application Publication
(10) Pub. No.: US 2011/0223256 A1
(43) Pub. Date: Sep. 15, 2011

(54) Method For Stabilizing Flavonoid Aqueous Dispersion

(75) Inventors: Naijie Zhang, Ridgefield, CT (US); Vincent E.A. Rinaldi, Bethel, CT (US); Peter S. Given, JR., Ridgefield, CT (US)

(73) Assignee: STOKELY-VAN CAMP, INC., Chicago, IL (US)

(21) Appl. No.: 13/036,161

(22) Filed: Feb. 28, 2011

Related U.S. Application Data
(60) Provisional application No. 61/312,694, filed on Mar. 11, 2010.

(51) Int. Cl. A61K 9/14; A61K 31/352; A61K 31/335; A61K 31/05; A61K 31/122; A61K 31/7048; A61P 33/06

(52) U.S. Cl. 424/493; 514/456; 514/450; 514/734; 514/679; 514/27

(57) ABSTRACT

A method for suspending microparticulated water insoluble bioactive compound in a beverage by incorporating solubilized or dispersed microparticulated compound and at least one dispersion stabilizer into a beverage. A composition comprising solubilized or dispersed microparticulated water insoluble bioactive compound and a dispersion stabilizer agent.

### FIELD OF THE INVENTION

The present invention relates generally to a method for dispersing and stabilizing water insoluble bioactive compounds in a liquid medium and a composition containing flavonoid and a suspension agent.

### BACKGROUND

Biologically active (bioactive) compounds such as flavonoids in general, are used as nutritional supplements to provide, for example, antioxidants. Most of bioactive compounds are highly rigid and crystalline and water insoluble. Quercetin, in particular, is considered a powerful antioxidant. A number of studies showed that quercetin is effective for the prevention of various diseases.

Flavonoids may be extracted from plants. For example, quercetin is a natural, plant-derived, flavonoid. In particular, quercetin is the aglycone form of a number of other flavonoid glycosides, such as rutin and quercitrin, found in citrus fruit, cranberries, blueberries, buckwheat, onions, and other vegetables, fruits, and green plants. The chemical structure of quercetin is illustrated below:

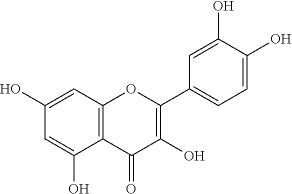

### 2-(3,4-dihydroxyphenyl)-3,5,7-trihydroxy-4H-chromen-4-one

It is desired to use flavonoids as a nutritional supplement in food products such as beverages. Often such flavonoids are difficult to disperse and mix into beverage products at efficacious concentrations. Often such flavonoids will simply settle to the bottom of the container holding the beverage. For example, quercetin is typically obtained as a powder and is insoluble in water. When added to liquid media, quercetin usually agglomerizes and settles to the bottom of the beverage, thereby resulting in a product that is not visually appealing to the consumer.

Therefore, a need exists in the food and beverage industry to provide the consumer with a food product containing bioactive compounds such as quercetin wherein the bioactive compound is stabilized in an aqueous suspension.

### BRIEF SUMMARY

The present invention relates to a method for stabilizing suspended quercetin or other suspended water insoluble bioactive compounds in a beverage and a composition for a beverage containing stabilized suspended quercetin or other water insoluble bioactive compounds.

### BRIEF DESCRIPTION OF THE DRAWINGS

FIGS. 1A and 1B compare amorphous quercetin and crystalline quercetin, respectively.

FIGS. 2A and 2B show the results of a quercetin stability study by FTIR and Carbon-13 NMR, respectively.

FIG. 3 shows samples of stable aqueous dispersions prepared in accordance with the present invention.

### DETAILED DESCRIPTION OF THE INVENTION

The present invention relates to a method for stabilizing suspended microparticulated water insoluble bioactive compounds such as quercetin particles in a liquid medium, such as a beverage, and further to a composition containing solubilized or dispersed compound particles and at least one dispersion stabilizer. Beverages, prepared with solubilized or dispersed a bioactive compound and a dispersion stabilizer, contain fine, stable dispersions.

The water insoluble bioactive compounds may be polyphenols, flavanoids, alkaloids, aldehyde, aryl ketone, benzofuranoid, benzopyranoid, diterpenoid, phenylpropanoid, polyketide, sesquiterpenoid, monoterpenoid, and/or may be derived from plants, herbs, or botanicals. See for example, Naturally Occurring Bioactive Compounds Edited by Mahendra Rai, Maria Cecillia Carpinella, 2006. Bioactive Compounds in Foods. Edited by John Gilbert and Hamide Z. Senyuva. Bioactive Compounds From Plants, Volume 154, John Wiley and Sons, 1990.

Suitable polyphenols include quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, anthocyanin, heptamethoxyflavone, curcumin, resveratrol, naringenin, tetramethoxyflavone, kaempferol, and rhoifolin.

Suitable flavonoids and other bioactive compounds include quercetin, flavonones, flavones, dihydroflavonols, flavonols, flavandiols, leucoanthocyanidins, flavonol glycosodes, flavonone glycosides, isoflavonoids, and neoflavonoids. In particular, the favonoids may be, but not limited to, quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, oolong tea polymerized polyphenol, anthocyanin, heptamethoxyflavone, daidzin, daidzein, biochaminn A, prunetin, genistin, glycitein, glycitin, genistein, 6,7,4′ trihydroxy isoflavone, morin, apigenin, vitexin, balcalein, apiin, cupressuflavone, datiscetin, diosmetin, fisetin, galangin, gossypetin, geraldol, hinokiflavone, primuletin, pratol, luteolin, myricetin, orientin, robinetin, quercetagetin, and hydroxy-4-flavone.

Suitable compounds derived from plants, herbs, or botanicals include pyrrolizldine, alkaloids, and artemisinin.

For ease of discussion, the application will be discussed in terms of quercetin. However, it is intended that the process steps and compositions apply to all suitable bioactive compounds described above and below.

In one aspect of the invention, quercetin is solubilized or dispersed prior to adding the quercetin to an aqueous solution containing a dispersion stabilizer.

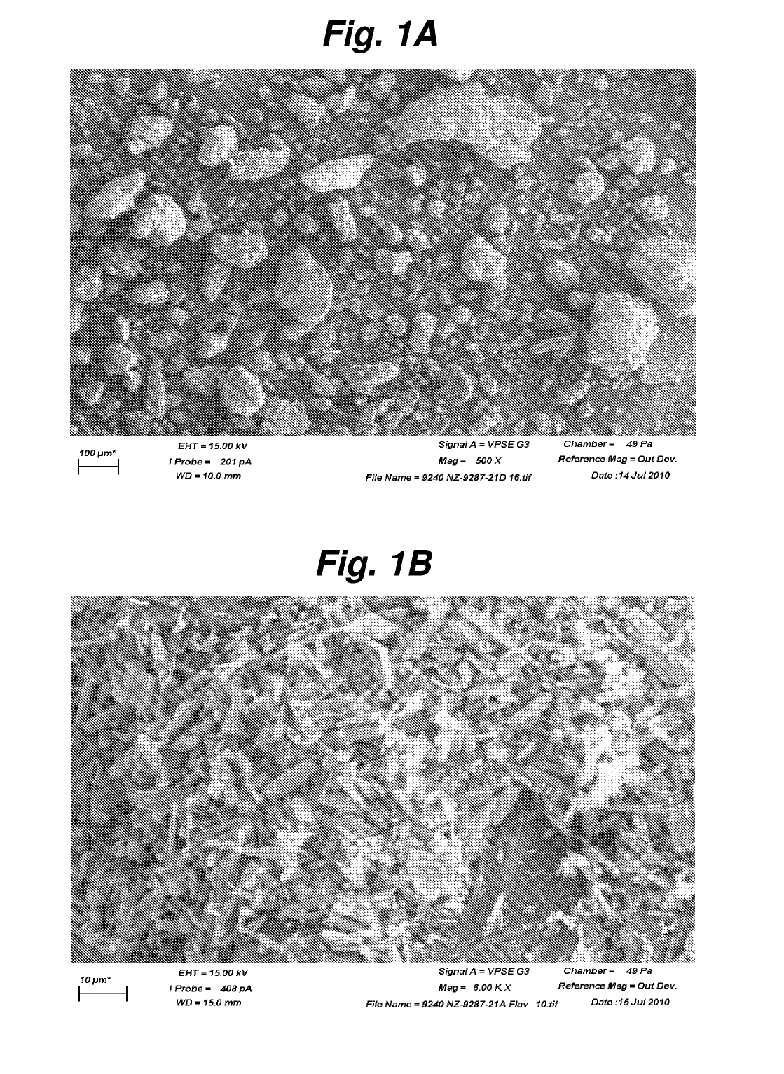

An aqueous dispersion of quercetin through solubilization reduces particle size, density, rigidity, and crystalline structure resulting in increasing dispersion stability. SEM results show dispersed quercetin in gellan gum has amorphous structure (FIG. 1A) compared to crystalline quercetin (FIG. 1B).

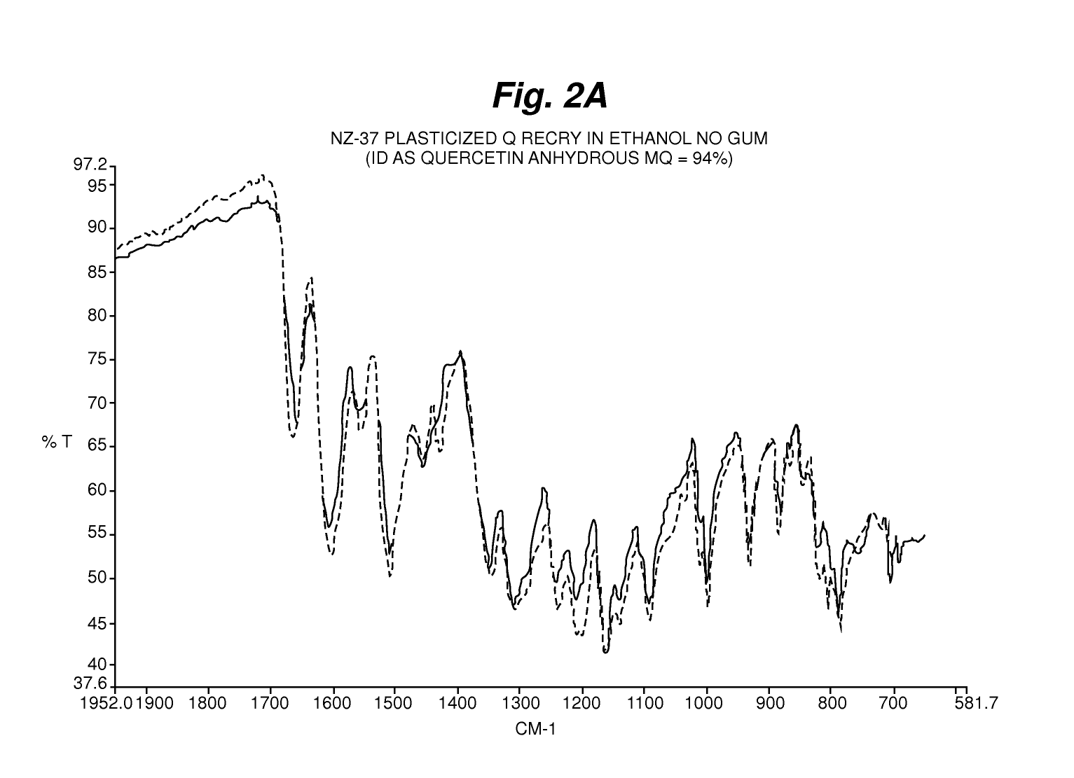

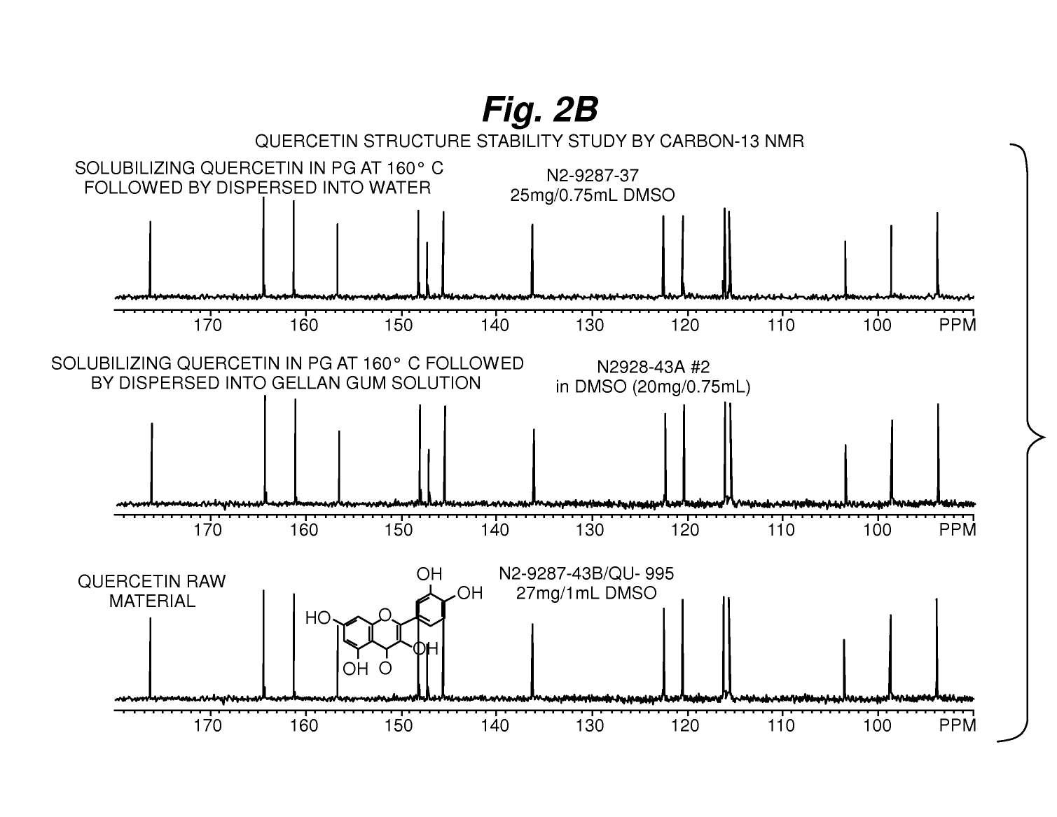

The chemical stability of quercetin during heating solubilizing process in polyols is determined by analytical characterization. Quercetin dispersion with or without gellan gum is isolated and purified from beverage. FIGS. 2A and 2B show that the spectra of FT-IR and carbon-13 NMR, respectively, of quercetin dispersions through solubilization are identical with that of starting material quercetin. The results indicate that there is no structure change of quercetin during heating solubilization process.

Quercetin is solubilized by dissolving quercetin in a) hot alcohol, such as hot ethanol or polyols, or b) an alkaline solution having a pH above 7 or c) a mixture of alcohol and alkaline solution, to form solubilized quercetin solution having 1 to 15 wt % quercetin. Suitable alcohols include, but not limited to, ethanol, isopropyl alcohol, isobutyl alcohol, and benzyl alcohol, and polyols such as glycerol and propylene glycol. Combinations of alcohols are also contemplated. Preferably ethanol is used in combination with an alkaline solution of quercetin.

When completely dissolved, the quercetin solution either in hot alcohol or alkaline is added into an aqueous solution containing a stabilizer with high shear mixing or agitation. The resulting quercetin comes out of solution resulting in fine particle dispersion, suspension. The alcohol concentration is generally between 85-99 wt. %.

In addition to solubilization, quercetin can be dispersed into the hot alcohol, such as hot ethanol or polyol, resulting in alcohol dispersion slurry in order to reduce quercetin particle size. Suitable alcohols include, but not limited to, ethanol, isopropyl alcohol, isobutyl alcohol, and benzyl alcohol, and polyols such as glycerol and propylene glycol. Combinations of alcohols are also contemplated.

Stable quercetin dispersion is obtained when hot alcohol dispersion slurry is added into an aqueous solution containing stabilizer under either high shear mixing or agitation conditions. The concentration of quercetin dispersion slurry in alcohol is in the range of 1-50 wt %.

The temperature of the hot alcohol for dissolving or dispersing the quercetin is between 50-200° C., generally 65-170° C.

The alkaline solution may be prepared from any suitable alkaline agent such as sodium hydroxide or potassium hydroxide. The pH of the alkaline solution is above 7, generally between 10 and 12.

After the quercetin is solubilized or dispersed, the quercetin is added to a solution containing at least one dispersion stabilizer. The solution is mixed or agitated to form stable quercetin dispersion. In particular, the solubilized or dispersed quercetin is added into a stabilizer-containing aqueous solution under mixing/agitation to form a dispersion containing from 0.1 to 10 weight % quercetin. The dispersion stabilizer is present in an amount sufficient to suspend the microparticulated quercetin in the beverage.

The aqueous solution containing dispersion stabilizer is maintained at a temperature below 35° C. and at pH less than 7, typically 3 to 5, during the addition of the solubilized alkaline quercetin solution. When quercetin is solubilized or dispersed in hot alcohol, the solution or dispersion slurry is cooled if necessary to between 50-130° C. prior to addition to stabilizer-containing aqueous solution.

Subsequently, water is added to dilute the concentrated dispersion to yield a stable quercetin dispersion having a concentration between 0.001-5.0 wt. %. The pH of the diluted dispersion is adjusted to less than 7.0, typically 2.5 to 6.

The at least one dispersion stabilizer can be a biopolymer or a modified polysaccharide such as gellan gum, pectin, carrageenan, ghatti gum, acacia gum, guar gum, xanthan gum, locust gum, agar, starch, alginate, cellulose, protein, hydrolyzed protein, modified starch, carboxyl methyl cellulose (CMC) or the combination thereof.

Preferably the biopolymers are charged polymers such as carboxyl-containing polymers and sulfate-containing polymers. It was discovered that anionic or cationic biopolymers such as pectin, gellan gum, carrageenan, gum arabic, ghatti gum, CMC, whey protein isolate showed better dispersion stability than non-ionic polymers. It is believed that quercetin absorbed on a charged polymer exhibits stable aqueous dispersion due to electrostatic, steric repulsion between the particles. There is no settling/aggregation after stored at ambient conditions. The inventors discovered that the length of time that the quercetin stays dispersed in the liquid media varies depending on the type(s) of dispersion stabilizer(s) used. For example, the quercetin can stay dispersed in a beverage for about 12 hours to about six months or longer, depending on the dispersion stabilizer(s) used.

Controlling pH during the process is critical to make stable dispersions and to suppress undesired color development. When an alkaline solution of quercetin is added into the stabilizer-containing aqueous solution under high shear mixing, pH of the dispersion should be maintained below 6, typically below 5, or 2.5 to 4. Otherwise, if the dispersion pH is alkaline, a dark brown color is developed and does not disappear even if the pH is lowered.

The quercetin is microparticulated. “Microparticulated” or “microparticulate” as used in the instant application means a small particle ranging in size from about 0.1 μm to about 50 μm with an average particle size below about 10 μm.

The microparticulated quercetin has an average particle size less than 10 microns, in particular less than 3 microns or less than 1 micron. For example, at least 90% of the particles have a particle size less than 50 microns and 80% of the particles have a particle size less than 3 microns.

The stability of quercetin aqueous dispersion is further improved by adding an organic compound with density less than 1.0 grams per cubic centimeter into an alcohol solution during solubilization of quercetin. Organic compound can be flavor ingredient such as limonene, terpene, citral, ethyl butyrate, ethyl acetate or oil such as coconut, cotton seed, olive, corn, palm, peanut, rapeseed, safflower, sesame, soja beans, sunflower, canola or combinations thereof. The concentration of organic compound in alcohol is from 0.001 to 50 wt. %.

As noted, the description focused on quercetin for ease of discussion; however, other water insoluble bioactive compounds were also investigated using the present invention dispersion method. Such bioactive compounds included curcumin, rutin, resveratrol, naringenin, hesperedin, tetramethoxyflavone (PMF), and artemisinin (anti malaria drug).

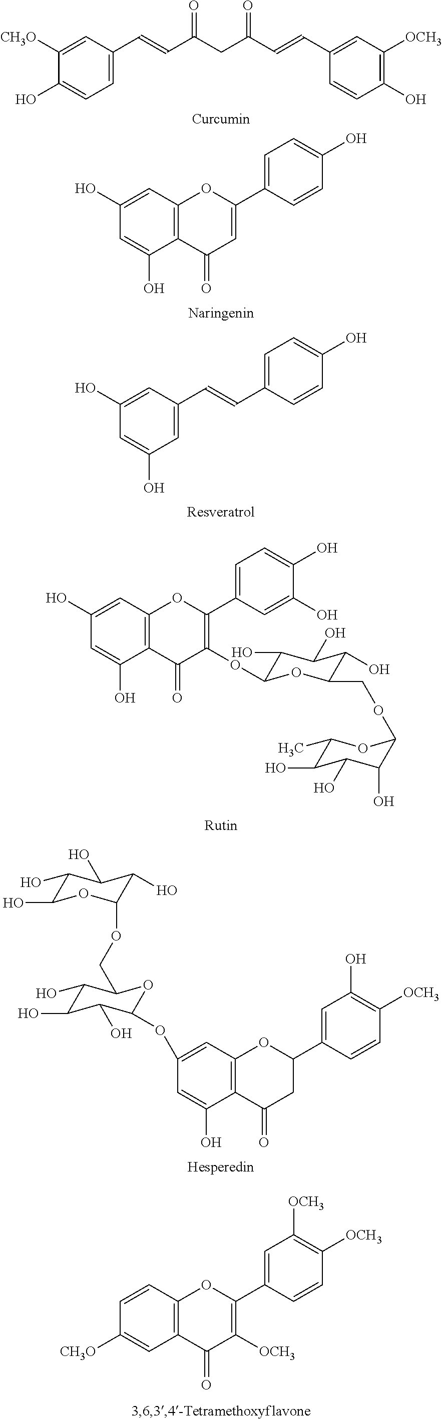

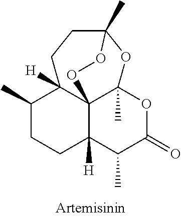

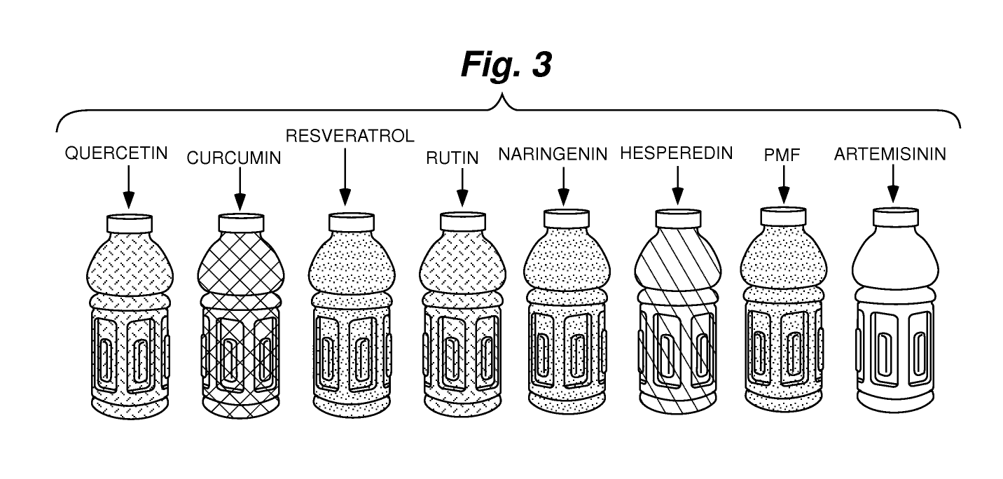

As set forth in the examples below, stable aqueous dispersions containing bioactive compounds were prepared by using the solubilization and stabilization process. The resulting dispersions were fine, homogeneous, and stable. There were no settling, phase separation, and precipitates on the side of the bottle (See FIG. 3 and Table 1). The results indicate that the presented dispersion method could be widely used to make stable water insoluble compound aqueous dispersion in different application areas.

TABLE 1
Water-Insoluble Bioactive Compounds
Aqueous Dispersion in 20 oz Beverage

| Compound | Amount (mg) | Shelf Life (22-25° C.) |
|---|---|---|
| Quercetin | 1000 | Stable after at least 6 months |
| Curcumin | 500 | Stable after at least 3 months |
| Rutin | 500 | Stable after at least 3 months |
| Resveratrol | 500 | Stable after at least 3 months |
| Naringenin | 500 | Stable after at least 3 months |
| Hesperedin | 500 | Stable after at least 3 months |
| Tetramethoxyflavone | 500 | Stable after at least 3 months |
| Artemisinin | 100 | Stable after at least 3 months |

The beverage may be any suitable beverage including, but not limited to, juices, carbonated soft drinks, water, dairy and isotonic beverages. One of ordinary skill in the art of the chemical and food sciences would recognize that any flavonoid may be used in accordance with the present invention.

Applicants also discovered that although the beverage appeared relatively viscous and/or thick upon adding the quercetin (or other water-insoluble bioactive compounds) to the beverage, by using the solubilized/dispersed quercetin in conjunction with the dispersion stabilizer, as disclosed herein, the beverage had a thin consistency upon consumption. This unexpected result, the thin consistency notwithstanding the viscous visual appearance, is also advantageous, as it leads to consumer likability and acceptance of the beverage.

Food-grade preservatives and acidulants may also be added to the solubilized water-insoluble bioactive compounds. Incorporating solubilized/dispersed water-insoluble bioactive compounds into the beverage results in improved suspension and dispersibility. It was further discovered that by incorporating the solubilized/dispersed water-insoluble bioactive compounds into the beverage, along with the dispersion stabilizer, that low concentration of the dispersion stabilizer may be added with a high concentration of water-insoluble bioactive compounds, and even though the water-insoluble bioactive compound is present in high concentrations and the beverage may look viscous, upon consumption it tastes thin and is easily ingested by the consumer.

The method of the present invention may also include one or more additional ingredients selected from the group consisting of carbohydrates, salts, salt blends, food-grade acids, flavors, colors, Vitamin B3, Vitamin C, non-nutritive and/or nutritive sweeteners and combinations of these ingredients.

Sweeteners of beverage embodiments of the invention include caloric carbohydrate sweeteners, natural high-potency sweeteners, synthetic high-potency sweeteners, other sweeteners, and combinations thereof. With the guidance provided herein, a suitable sweetening system (whether a single compound or combination thereof) can be selected.

Examples of suitable caloric carbohydrate sweeteners include sucrose, fructose, glucose, erythritol, maltitol, lactitol, sorbitol, mannitol, xylitol, D-tagatose, trehalose, galactose, rhamnose, cyclodextrin (e.g., α-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, glucuronic acid, gluconic acid, glucono-lactone, abequose, galactosamine, xylo-oligosaccharides (xylotriose, xylobiose and the like), gentio-oligoscaccharides (gentiobiose, gentiotriose, gentiotetraose and the like), galacto-oligosaccharides, sorbose, nigero-oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), lactulose, melibiose, raffinose, rhamnose, ribose, isomerized liquid sugars such as high fructose corn/starch syrup (e.g., HFCS55, HFCS42, or HFCS90), coupling sugars, soybean oligosaccharides, and glucose syrup.

Other sweeteners suitable for use in embodiments provided herein include natural, synthetic, and other high-potency sweeteners. As used herein, the phrases “natural high-potency sweetener,” “NHPS,” “NHPS composition,” and “natural high-potency sweetener composition” are synonymous. “NHPS” means any sweetener found in nature which may be in raw, extracted, purified, treated enzymatically, or any other form, singularly or in combination thereof and characteristically has a sweetness potency greater than sucrose, fructose, or glucose, yet has fewer calories. Non-limiting examples of NHPS's suitable for embodiments of this invention include rebaudioside A, rebaudioside B, rebaudioside C (dulcoside B), rebaudioside D, rebaudioside E, rebaudioside F, dulcoside A, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, and cyclocarioside I.

NHPS also includes modified NHPS's. Modified NHPS's include NHPS's which have been altered naturally. For example, a modified NHPS includes, but is not limited to, NHPS's which have been fermented, contacted with enzyme, or derivatized or substituted on the NHPS. In one embodiment, at least one modified NHPS may be used in combination with at least one NHPS. In another embodiment, at least one modified NHPS may be used without a NHPS. Thus, modified NHPS's may be substituted for a NHPS or may be used in combination with NHPS's for any of the embodiments described herein. For the sake of brevity, however, in the description of embodiments of this invention, a modified NHPS is not expressly described as an alternative to an unmodified NHPS, but it should be understood that modified NHPS's can be substituted for NHPS's in any embodiment disclosed herein.

As used herein, the phrase “synthetic sweetener” refers to any composition that is not found in nature and is a high potency sweetener. Non-limiting examples of synthetic sweeteners suitable for embodiments of this invention include sucralose, acesulfame potassium (acesulfame K or aceK) or other salts, aspartame, alitame, saccharin, neohesperidin dihydrochalcone, cyclamate, neotame, N-[3-(3-hydroxy-4-methoxyphenyl)propyl]-L-α-aspartyl]-phenylalanine 1-methyl ester, N-[3-(3-hydroxy-4-methoxyphenyl)-3-methylbutyl]-L-α-aspartyl]-L-phenylalanine 1-methyl ester, N-[3-(3-methoxy-4-hydroxyphenyl)propyl]-L-α-aspartyl]-L-phenylalanine 1-methyl ester, and salts thereof.

The method described herein is advantageous as it successfully suspends water-insoluble bioactive compounds such as quercetin in the beverage and thereby deters settling of the water-insoluble bioactive compounds to the bottom of the beverage's packaging.

The present invention also relates to compositions comprising solubilized or dispersed water-insoluble bioactive compounds and at least one dispersion stabilizer. In one aspect of this invention, about 90% of the microparticulated water-insoluble bioactive compounds is below about 50 microns and the at least one dispersion stabilizer is present in an amount sufficient to suspend the water-insoluble bioactive compounds in a liquid medium.

The stability of water-insoluble bioactive compound aqueous dispersion is further improved by adding an organic compound with density less than 1.0 grams per cubic centimeter into alcohol solution during solubilization of the water-insoluble bioactive compound. Suitable organic compounds can be flavor ingredient such as limonene, terpene, citral, ethyl butyrate, ethyl acetate or oil such as coconut, cotton seed, olive, corn, palm, peanut, rapeseed, safflower, sesame, soja beans, sunflower, canola or the combination. The concentration of organic compound in alcohol is between 0.1-30 wt. %.

The following examples are specific embodiments of the present invention, but are not intended to limit the invention.

### Example 1

A quercetin solution was prepared by dissolving 1.0 g quercetin to 20 g glycerol at temperature 150° C. Then the solubilized quercetin solution was slowly added to an aqueous solution containing gellan gum under high mixing at pH 5. A homogeneous dispersion containing 0.5% quercetin was obtained.

The concentrated quercetin dispersion was added to the beverage and suspended in the beverage. Additional ingredients were added in the concentrations (w/w) listed below to make an isotonic beverage containing quercetin. The pH was about 3.5. The pH range of the resultant isotonic beverage may be about 2.5-4.5.

| | Amount |
|---|---|
| Ingredient | (% by wt.) |
| Water | 92.16% |
| Liquid Sucrose | 2.963% |
| Salt Blend | 0.176% |
| Quercetin | 0.2% |
| Gellan gum | 0.03% |
| Citric Acid | 0.180% |
| Glycerol | 4% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 2

Quercetin (1.0 g) was dissolved in 20 g propylene glycol at 150° C. Then, quercetin solution was slowly added into gellan gum-containing aqueous solution under high shear mixing at pH 5. The quercetin dispersion was added to beverage. Additional ingredients were added in the concentrations (w/w) listed below to make an isotonic beverage containing quercetin. The pH range of the resultant isotonic beverage was 3.5.

| | Amount |
|---|---|
| Ingredient | (% by wt.) |
| Water | 92.16% |
| Liquid Sucrose | 2.963% |
| Salt Blend | 0.176% |
| Quercetin | 0.2% |
| Gellan gum | 0.03% |
| Citric Acid | 0.180% |
| Propylene Glycol | 4% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 3

Quercetin (1.0 g) was dissolved in 10 g glycerol and 10 g propylene glycol at 150° C. Then, quercetin polyol solution was slowly added into gellan gum-containing aqueous solution under high mixing at pH 5. The quercetin dispersion was added to beverage. Additional ingredients were added in the concentrations (w/w) listed below to make an isotonic beverage containing quercetin. The pH range of the resultant isotonic beverage was 3.5.

| | Amount |
|---|---|
| Ingredient | (% by wt.) |
| Water | 92.16% |
| Liquid Sucrose | 2.963% |
| Salt Blend | 0.176% |
| Quercetin | 0.2% |
| Gellan gum | 0.03% |
| Citric Acid | 0.180% |
| Glycerol | 2% |
| Propylene Glycol | 2% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 4

The quercetin solution was prepared by dissolving 0.5 g quercetin into 10 g sodium hydroxide (0.5 N) at room temperature. Then, quercetin alkaline solution was slowly added to an aqueous solution containing carrageenan under homogenization at pH 5. The dispersion pH was controlled below 7 during addition of quercetin alkaline solution. A homogeneous dispersion containing 0.5% quercetin is obtained. The concentrated quercetin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.37% |
| Citric Acid | 0.050% |
| Phosphoric Acid | 0.016% |
| Vitamin B3 | 0.005% |
| Ascorbic Acid | 0.109% |
| Carrageenan | 0.1% |
| Quercetin | 0.1% |
| Salt Blend | 0.176% |
| Reb A | 0.025% |
| Orange Flavor | 0.050% |
| Total | 100.000% |

Alternatively, or in addition to, the salt blend listed above, the beverage may contain sea salt to produce a natural zero-calorie isotonic beverage.

### Example 5

Quercetin (0.5 g) was dispersed and partially solubilized in 2.5 g ethanol at 70° C. Then, the partially solubilized, dispersed quercetin slurry was slowly added into ghatti gum-containing aqueous solution under homogenization at pH 5. The quercetin dispersion was added to beverage. Additional ingredients were added in the concentrations (w/w) listed below to make an isotonic beverage containing quercetin. The pH was 3.5.

| | Amount |
|---|---|
| Ingredient | (% by wt.) |
| Water | 95.59% |
| Liquid Sucrose | 2.963% |
| Ethanol | 0.5 |
| Salt Blend | 0.176% |
| Quercetin | 0.1% |
| Ghatti Gum | 0.2% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 6

Quercetin (0.5 g) was dissolved in 4 g propylene glycol at 140° C. Then, the quercetin solution was slowly added into gum arabic-containing aqueous solution under homogenization at pH 5. The quercetin dispersion was added to beverage. Additional ingredients were added in the concentrations (w/w) listed below to make an isotonic beverage containing quercetin. The pH range of the resultant isotonic beverage was 3.5.

| | Amount |
|---|---|
| Ingredient | (% by wt.) |
| Water | 95.29% |
| Liquid Sucrose | 2.963% |
| Salt Blend | 0.176% |
| Quercetin | 0.1% |
| Gum Arabic | 0.2% |
| Propylene Glycol | 0.8% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 7

The quercetin solution was prepared by dissolving 0.5 g quercetin to 10 g glycerol and 0.1 g limonene at temperature 120° C. Then the solubilized quercetin solution was slowly added to an aqueous solution containing gum arabic under homogenization at pH 5. A homogeneous dispersion containing 0.5% quercetin was obtained. The concentrated quercetin dispersion was added to the beverage and suspended in the beverage. Additional ingredients were added in the concentrations (w/w) listed below to make an isotonic beverage containing quercetin. The pH was 3.0.

| | Amount |
|---|---|
| Ingredient | (% by wt.) |
| Water | 94.17% |
| Liquid Sucrose | 2.963% |
| Salt Blend | 0.176% |
| Quercetin | 0.1% |
| Gum Arabic | 0.1% |
| Citric Acid | 0.180% |
| Glycerol | 2% |
| limonene | 0.02% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 8

The quercetin solution was prepared by dissolving 0.5 g quercetin to 10 g glycerol and 1.0 g canola oil at temperature 120° C. Then the solubilized quercetin solution was slowly added to an aqueous solution containing modified starch (purity gum 2000) under homogenization at pH 5. A homogeneous dispersion containing 0.5% quercetin was obtained. The concentrated quercetin dispersion was added to the beverage and suspended in the beverage. Additional ingredients were added in the concentrations (w/w) listed below to make an isotonic beverage containing quercetin. The pH was 3.5.

| | Amount |
|---|---|
| Ingredient | (% by wt.) |
| Water | 93.59% |
| Liquid Sucrose | 2.963% |
| Salt Blend | 0.176% |
| Quercetin | 0.1% |
| Purity Gum 2000 | 0.5% |
| Citric Acid | 0.180% |
| Glycerol | 2% |
| Canola Oil | 0.2% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 9

Quercetin (0.5 g) was dissolved in 10 g glycerol at 150° C. Then, the quercetin solution was slowly added into pectin-containing aqueous solution under high shear mixing at pH 5. The quercetin dispersion was added to beverage. Additional ingredients were added in the concentrations (w/w) listed below to make an isotonic beverage containing quercetin. The pH range of the resultant isotonic beverage was 3.5.

| | Amount |
|---|---|
| Ingredient | (% by wt.) |
| Water | 94.09% |
| Liquid Sucrose | 2.963% |
| Salt Blend | 0.176% |
| Quercetin | 0.1% |
| Pectin | 0.2% |
| Citric Acid | 0.180% |
| Glycerol | 2% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 10

Curcumin (0.5 g) was dissolved in 24 g glycerol at 150° C. Then, curcumin solution was slowly added into gellan gum-containing aqueous solution under high shear mixing at pH 5. The curcumin dispersion was added to beverage. Additional ingredients were added in the concentrations (w/w) listed below to make an isotonic beverage containing curcumin. The pH range of the resultant isotonic beverage was 3.5.

| | Amount |
|---|---|
| Ingredient | (% by wt.) |
| Water | 92.5% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Curcumin | 0.0833% |
| Sodium Benzoate | 0.1% |
| Total | 100.000% |

### Example 11

Rutin hydrate (0.5 g) was dissolved in 24 g glycerol at 150° C. Then, Rutin solution was slowly added into gellan gum-containing aqueous solution under high shear mixing at pH 5. The Rutin dispersion was added to beverage. Additional ingredients were added in the concentrations (w/w) listed below to make an isotonic beverage containing Rutin. The pH range of the resultant isotonic beverage was 3.5.

| | Amount |
|---|---|
| Ingredient | (% by wt.) |
| Water | 92.5% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Rutin | 0.0833% |
| Sodium Benzoate | 0.1% |
| Total | 100.000% |

### Example 12

Resveratrol (0.5 g) was dissolved in 24 g glycerol at 150° C. Then, resveratrol solution was slowly added into gellan gum-containing aqueous solution under high shear mixing at pH 5. The resveratrol dispersion was added to beverage. Additional ingredients were added in the concentrations (w/w) listed below to make an isotonic beverage containing resveratrol. The pH range of the resultant isotonic beverage was 3.5.

| | Amount |
|---|---|
| Ingredient | (% by wt.) |
| Water | 92.5% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Resveratrol | 0.0833% |
| Sodium Benzoate | 0.1% |
| Total | 100.000% |

### Example 13

Artemisinin (0.1 g) was dissolved in 24 g glycerol at 150° C. Then, artemisinin solution was slowly added into gellan gum-containing aqueous solution under high shear mixing at pH 5. The artemisinin dispersion was added to beverage. Additional ingredients were added in the concentrations (w/w) listed below to make an isotonic beverage containing artemisinin. The pH range of the resultant isotonic beverage was 3.5.

| | Amount |
|---|---|
| Ingredient | (% by wt.) |
| Water | 92.57% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Artemisinin | 0.017% |
| Sodium Benzoate | 0.1% |
| Total | 100.000% |

### Example 14

Naringenin (0.5 g) was dissolved in 24 g glycerol at 150° C. Then, naringenin solution was slowly added into gellan gum-containing aqueous solution under high shear mixing at pH 5. The naringenin dispersion was added to beverage. Additional ingredients were added in the concentrations (w/w) listed below to make an isotonic beverage containing naringenin. The pH range of the resultant isotonic beverage was 3.5.

| | Amount |
|---|---|
| Ingredient | (% by wt.) |
| Water | 92.5% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Naringenin | 0.0833% |
| Sodium Benzoate | 0.1% |
| Total | 100.000% |

### Example 15

Hesperedin (0.5 g) was dissolved in 24 g glycerol at 150° C. Then, hesperedin solution was slowly added into gellan gum-containing aqueous solution under high shear mixing at pH 5. The hesperedin dispersion was added to beverage. Additional ingredients were added in the concentrations (w/w) listed below to make an isotonic beverage containing hesperedin. The pH range of the resultant isotonic beverage was 3.5.

| | Amount |
|---|---|
| Ingredient | (% by wt.) |
| Water | 92.5% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Hesperedin | 0.0833% |
| Sodium Benzoate | 0.1% |
| Total | 100.000% |

### Example 16

3,6,3′,4′-Tetramethoxyflavone (0.5 g) was dissolved in 24 g glycerol at 150° C. Then, tetramethoxyflavone solution was slowly added into gellan gum-containing aqueous solution under high shear mixing at pH 5. The tetramethoxyflavone dispersion was added to beverage. Additional ingredients were added in the concentrations (w/w) listed below to make an isotonic beverage containing tetramethoxyflavone. The pH range of the resultant isotonic beverage was 3.5.

| | Amount |
|---|---|
| Ingredient | (% by wt.) |
| Water | 92.5% |
| Sucrose | 1.93% |
| Sodium Citrate | 0.13% |
| Monopotassium Phosphate | 0.04% |
| Citric Acid | 0.187% |
| Reb A | 0.015% |
| Erythritol | 0.885% |
| Ascorbic Acid | 0.103% |
| Gellan Gum | 0.03% |
| Glycerol | 4% |
| Tetramethoxyflavone | 0.0833% |
| Sodium Benzoate | 0.1% |
| Total | 100.000% |

This invention may be embodied in other specific forms without departing from the spirit or essential characteristics thereof. The foregoing embodiments, therefore, are to be considered in all respects illustrative rather than limiting the invention described herein. The scope of the invention is thus indicated by the appended claims, rather than by the foregoing description, and all changes that come within the meaning and range of equivalency of the claims are intended to be embraced therein.

We claim:

1. A method for dispersing and suspending microparticulated water insoluble bioactive compound particles in a beverage comprising the steps of a) solubilizing water insoluble bioactive compound in a hot alcohol solution, or in an alkaline solution, or combination thereof or b) dispersing the water insoluble bioactive compound in a hot alcohol solution; and c) introducing the solubilized or dispersed bioactive compound into an aqueous solution containing at least one dispersion stabilizer; wherein the dispersion stabilizer is present in an amount sufficient to suspend the water insoluble bioactive compound in the beverage, wherein the suspended water insoluble bioactive compound comprises microparticulated particles.

2. The method of claim 1 wherein the water insoluble bioactive compound is a polyphenol.

3. The method of claim 1 wherein the water insoluble bioactive compound is a flavonoid.

4. The method of claim 3 wherein the flavonoid is selected from the group consisting of quercetin, flavonones, flavones, dihydroflavonols, flavonols, flavandiols, leucoanthocyanidins, flavonol glycosodes, flavonone glycosides, isoflavonoids, and neoflavonoids.

5. The method of claim 1 wherein the water insoluble bioactive compound is selected from the group consisting of quercetin, flavonones, flavones, dihydroflavonols, flavonols, flavandiols, leucoanthocyanidins, flavonol glycosodes, flavonone glycosides, isoflavonoids, and neoflavonoids. In particular, the favonoids may be, but not limited to, quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, oolong tea polymerized polyphenol, anthocyanin, heptamethoxyflavone, daidzin, daidzein, biochaminn A, prunetin, genistin, glycitein, glycitin, genistein, 6,7,4′ trihydroxy isoflavone, morin, apigenin, vitexin, balcalein, apiin, cupressuflavone, datiscetin, diosmetin, fisetin, galangin, gossypetin, geraldol, hinokiflavone, primuletin, pratol, luteolin, myricetin, orientin, robinetin, quercetagetin, and hydroxy-4-flavone.

6. The method of claim 1 wherein the water insoluble bioactive compound is extracted from plants, herbs, or botanicals.

7. The method of claim 1 wherein the water insoluble bioactive compound is selected from the group consisting of curcumin, rutin, resveratrol, naringenin, hesperedin, tetramethoxyflavone (PMF), and artemisinin.

8. The method of claim 1 wherein the water insoluble bioactive compound is quercetin.

9. The method of claim 1 wherein the at least one dispersion stabilizer is a n anionic or cationic biopolymer or a modified polysaccharide selected from the group consisting of gellan gum, pectin, guar gum, xanthan gum, acacia gum, locust gum, agar, starch, ghatti gum, carrageenan, alginate, cellulose, protein, hydrolyzed protein, modified starch, carboxyl methyl cellulose, or the combination thereof.

10. The method of claim 9 wherein the at least one dispersion stabilizer is a gellan gum, pectin, gum arabic, ghatti gum, carrageenan, alginate, CMC, whey protein isolate, or combination thereof.

11. The method of claim 1 wherein the dispersion stabilizer is present in the beverage in a concentration of about 0.001-5.0 wt. % based on total weight of the beverage.

12. The method of claim 1 wherein the beverage has a pH of less than 6.

13. The method of claim 13 wherein the pH of the beverage is about 2.5 to about 4.5.

14. The method of claim 1 wherein the alcohol is a polyol.

15. The method of claim 1 wherein alcohol is selected from the group consisting of ethanol, benzyl alcohol, isopropyl alcohol, isobutyl alcohol, glycerol, and propylene glycol.

16. The method of claim 1 wherein the water insoluble bioactive compound is solubilized in a hot glycerol or hot propylene glycol or hot ethanol.

17. The method of claim 1 wherein the water insoluble bioactive compound is solubilized in an alkaline solution at a pH of from 10 to 12.

18. The method of claim 1 wherein the alkaline solution comprises sodium hydroxide or potassium hydroxide.

19. The method of claim 1 wherein the hot alcohol solution further comprises an organic compound selected from the group consisting of limonene, terpene, citral, ethyl butyrate, ethyl acetate, coconut oil, cotton seed oil, olive oil, corn oil, palm oil, peanut oil, rapeseed oil, safflower oil, sesame oil, soja beans oil, sunflower oil, canola oil, or combinations thereof.

20. The method of claim 20 wherein the concentration of organic compound in alcohol is from 0.1 to 30 wt. %.

21. The method of claim 1 wherein the beverage further comprises at least one additional ingredient selected from the group consisting of carbohydrates, salts, salt blends, food-grade acids, flavors, colors, Vitamin B3, Vitamin C, sweeteners and combinations thereof.

22. The method of claim 22 wherein the beverage further comprises salt in a concentration of about 0.1-0.3 wt % based on the total weight of the beverage and carbohydrate in a concentration of about 1-3 wt % based on the total weight of the beverage.

23. The method of claim 22 wherein the beverage further comprises at least one sweetener selected from the group consisting of stevia, monatin and combinations thereof.

24. The method of claim 1 wherein the hot alcohol is at a temperature of 50 to 200° C.

25. A beverage prepared in accordance with the method of claim 1.

26. A method for suspending flavonoid particles in a beverage comprising combining at least one dispersion stabilizer and solubilized or dispersed microparticulated flavonoid, wherein the dispersion stabilizer is present in an amount sufficient to suspend the microparticulated flavonoid in the beverage, and wherein the flavonoid is solubilized or dispersed in hot glycerol, hot propylene glycol, hot ethanol, an alkaline solution, or combination thereof.

27. The method of claim 26 wherein the flavonoid is quercetin.

28. The method of claim 26 wherein the at least one dispersion stabilizer is an anionic or cationic biopolymer.

29. The method of claim 28 wherein the at least one dispersion stabilizer is a gellan gum, gum arabic, pectin, carrageenan, ghatti gum, alginate, CMC, whey protein isolate, or combination thereof.

30. The method of claim 26, wherein the particles are microparticulated.

31. A composition for a comestible comprising solubilized, dispersed microparticulated flavonoid and at least one dispersion agent, wherein about 90% of the flavonoid has a particle size below about 50 microns and the at least one dispersion agent is present in an amount sufficient to suspend the flavonoid in a liquid medium; wherein the microparticulated flavonoid is solubilized or dispersed in hot glycerol, hot propylene glycol, hot ethanol, an alkaline solution, or combinations thereof.

32. The composition for a comestible of claim 30 wherein the flavonoid is quercetin.
